(19) United States
(12) Patent Application Publication
(10) Pub. No.: US 2024/0359006 A1
(43) Pub. Date: Oct. 31, 2024

(54) ELECTRICAL MODULATION FOR THE TREATMENT OF BRAIN TUMORS

(71) Applicant: Northwestern University, Evanston, IL (US)

(72) Inventor: Timothy L. Sita, Kildeer, IL (US)

(21) Appl. No.: 18/648,764

(22) Filed: Apr. 29, 2024

Related U.S. Application Data
(60) Provisional application No. 63/499,115, filed on Apr. 28, 2023.

(51) Int. Cl. A61N 1/36; A61N 1/04; A61N 1/20

(52) U.S. Cl. CPC A61N 1/36002; A61N 1/0408; A61N 1/205; A61N 1/36034

(57) ABSTRACT

Electrical modulation provides treatment of brain tumors, such as glioblastoma. An electrical stimulus may be applied to the brain of a patient with a brain tumor (e.g., primary or metastatic brain tumor in upfront, adjuvant, or recurrent treatment setting) via bitemporal and/or unitemporal electrodes. The electrical modulation acts on brain tumor networks to slow tumor growth and increase survivability of the patient. The electrical modulation also transiently permits passage of therapeutics across the blood-brain barrier. The electrical modulation also sensitizes brain tumors to radiation therapy, immunotherapy, and chemotherapy.

### BACKGROUND

Glioblastoma (GBM) is a type of incurable brain tumor with a very poor prognosis. Unfortunately, most patients will not survive longer than two years following diagnosis despite an aggressive combination of surgery, radiation, and chemotherapy. GBM and many other types of brain tumors (including metastatic and other aggressive primary central nervous system tumors) are electrical in nature and hijack our normal brain's electrical circuitry to fuel their growth and spread throughout the brain. Additionally, brain tumors that are electrically connected to themselves and to our normal brain's electrical networks are more resistant to radiation and chemotherapy.

### SUMMARY OF THE DISCLOSURE

It is an aspect of the present disclosure to provide a method for electrical modulation of a glioblastoma (GBM) in a subject. The method includes receiving, by a neuromodulation device, stimulation settings determined to generate an electrical stimulation that modulates one or more genes associated with a GBM signaling pathway. Electrical stimulation is delivered to the subject, via one or more electrodes connected to the neuromodulation device, using the stimulation settings. The electrical stimulation provides a therapeutic effect in the subject by modulating the one or more genes associated with the GBM signaling pathway. Other embodiments of this aspect include corresponding systems (e.g., computer systems, controllers), programs, algorithms, and/or modules, each configured to perform the steps of the methods.

Implementations may also include one or more of the following features. The stimulation settings may include at least one of a voltage, a current level, a frequency, a pulse width, or a duration of the electrical stimulation. The current level can be selected from a range of 20-100 mA. The frequency can be selected from a range of 10-100 Hz. The pulse width can be selected from a range of 0.5-2.0 ms. The duration of the electrical stimulation can be selected from a range of 0.5-2.0 s.

In some aspects, the method may include providing at least one additional treatment before, during, or after the electrical stimulation. The at least one additional treatment can be at least one of radiation therapy, immunotherapy, or chemotherapy.

In some aspects, the stimulation settings can be predetermined to induce a therapeutic seizure in the subject having a duration of between 10 and 120 seconds, such that the therapeutic seizure induces modulating the one or more genes associated with the GBM signaling pathway.

In some aspects, the GBM signaling pathway may include one or more GBM-associated electrical network signaling pathways. Additionally or alternatively, the GBM signaling pathway may include one or more GBM neuronal activity drivers. Additionally or alternatively, the GBM signal pathway may include one or more GBM network-associated signaling proteins. The one or more genes may include at least one of neuronal growth associated protein 43 (GAP43) or neuroligin 3 (NLGN3). Additionally or alternatively, the one or more genes may include at least one of GRIA4, GRIK1, GRIN1, or GRM4. Modulating the one or more genes may include downregulating the one or more genes.

It is another aspect of the present disclosure to provide a system for electrical modulation of brain tumors. The system also includes one or more electrodes to deliver electrical stimulation to a subject; a memory having stored thereon electrical stimulation parameters predetermined to induce a therapeutic seizure in a subject having a duration between 10 and 120 seconds; and a processor to drive the one or more electrodes using the electrical stimulation parameters stored on the memory.

### BRIEF DESCRIPTION OF THE DRAWINGS

FIG. 1 is a flowchart of an example method for delivering electrical modulation to a tumor to provide a therapeutic effect in a subject.

FIG. 2 illustrates an example of downregulation of GAP43 in glioblastoma (GBM) following delivery of electrical modulation.

FIG. 3 illustrates an example of downregulation of NLGN3 in GBM following delivery of electrical modulation.

FIGS. 4A-4D illustrate examples of downregulation of glutamate receptors GRIA4 (FIG. 4A), GRIK1 (FIG. 4B), GRIN1 (FIG. 4C), and GRM4 (FIG. 4D) following delivery of electrical modulation.

FIG. 5 is a data plot illustrating increased intratumoral microglia cells following electrical modulation.

FIG. 6 is a data plot illustrating increased intratumoral tumor-associated myeloid cells following electrical modulation.

FIG. 7 shows data plots illustrating increased intratumoral CD4+ and CD8+ T-cells following electrical modulation.

FIG. 8 shows data plots illustrating increased intratumoral innate inflammatory markers following electrical modulation.

FIG. 9 shows example histopathology images depicting increased intratumoral CD3+ T-cells following electrical modulation.

FIG. 10 shows an example of tumor bioluminescence, H&E stained images, and tumor weights after taken before and after delivery of electrical modulation to provide a therapeutic effect.

FIGS. 11A and 11B show electrical modulation reliably inducing seizure in glioma-bearing mice. FIG. 11A shows C57BL6 mice bearing CT-2A xenografts underwent EEG implantation. FIG. 11B shows mice were subjected to electrical modulation using a Ugo Basile model 57800 machine (Stoelting Co.) at a current level of 80 mA, frequency of 50 Hz, pulse width of 0.5 ms, and shock duration of 1.0 s. These settings reproducibly generated EEG readings consistent with seizure activity lasting an average of 28 seconds (range 20 to 44 seconds).

FIGS. 12A and 12B show data indicating that repeated electrical modulation slowing tumor progression and prolonging overall survival in C57BL6 mice bearing CT-2A-Luc xenografts. FIG. 12A shows radiance in photons per second from CT-2A-Luc glioma-bearing mice randomized to receive electrical modulation via ear-clip electrodes or sham treatment daily up to five times per week. FIG. 12B shows the overall survival of CT-2A-Luc glioma-bearing mice randomized to receive electrical modulation via ear-clip electrodes or sham treatment daily up to five times per week.

FIGS. 13A and 13B show data indicating that repeated electrical modulation with leg extension prolongs overall survival in C57BL6 mice bearing CT-2A-Luc xenografts. FIG. 13A shows overall survival of CT-2A-Luc glioma-bearing mice randomized to receive electrical modulation via ear-clip electrodes with at least 75% of seizures clinically demonstrating bilateral hind limb extension (EM w/LE) or sham treatment daily up to five times per week. FIG. 13B shows Pearson correlation between percent of electrical modulation sessions with limb extension and days survived.

FIG. 14 shows fluorescence of C57BL6 brains treated with one session of electrical modulation or sham treatment delivered five minutes after tail vein injection of sodium fluorescein (40 mg/kg). Mice were perfused with PBS 30 minutes following tail vein injection and brains harvested for fluorescent analysis at excitation wavelength 465 nm and emission wavelength 530 nm.

FIG. 15 shows a gene ontological analysis of electrical modulation-downregulated tumor RNA sequencing genes.

FIG. 16 illustrates increased perfusion following electrical modulation measured using multiparametric magnetic resonance imaging (MRI).

FIG. 17 illustrates increased oxygenation following electrical modulation measured using multiparametric MRI.

FIG. 18 is a block diagram of an example controller that can implement the methods described in the present disclosure.

FIG. 19a block diagram of an example system for delivering electrical modulation to a brain tumor, such as a glioblastoma.

FIG. 20 is a block diagram of example components that can implement the system of FIG. 19.

### DETAILED DESCRIPTION

Described here are systems and methods for using neuromodulation, such as electrical modulation, to provide treatment of brain tumors. As a non-limiting example, under general anesthesia, an electrical stimulus may be applied to the brain of a patient with a brain tumor (e.g., primary or metastatic brain tumor in upfront, adjuvant, or recurrent treatment setting) via bitemporal and/or unitemporal electrodes. The electrical stimulus is configured as described in the present disclosure such that it is a capable of inducing a therapeutic seizure that clinically lasts 10-120 seconds in duration. The strength of the stimulus needed (e.g., current, voltage, shock frequency, shock duration) will vary between patients and possibly between sessions of the same patient depending on depth of anesthesia, size of brain tumor or tumor resection cavity, other medications that influence seizure threshold, etc. The induced seizures may be monitored clinically and/or using EEG, and may be stopped using abortive medical therapy when prolonged in duration.

Currently approved therapeutics do not attempt to target and dissemble electrical brain tumor networks through application of a robust electrical stimulus potent enough to induce an electrical network override, resetting and disconnecting brain tumor networks from themselves and normal healthy brain tissue.

It is an advantage of the present disclosure that the described electrical modulation may act on brain tumor networks to slow tumor growth and improve survival in the upfront, adjuvant, and recurrent treatment setting. Additionally or alternatively, the described electrical modulation may transiently permit passage of therapeutics across the blood-brain barrier, including chemotherapeutics, immunotherapies, and targeted therapies with traditionally poor central nervous system penetrance. In still other aspects, the described electrical modulation may sensitize brain tumors to radiation therapy, immunotherapy, and chemotherapy, synergizing with and enhancing current standard-of-care therapeutic options.

To disassemble electrical tumor networks and stop electrical signaling from fueling therapeutic resistance, the disclosed techniques may use electrical modulation (EM), which can be administered through the application of an electrical stimulus to GBM-bearing subjects. EM provides an electrical override to pathologic GBM electrical networks akin to how defibrillation resets the electric current of a dysrhythmic heart.

Preliminary data described in the present disclosure demonstrate that this technique can slow tumor progression and improve overall survival in mice bearing GBM patient-derived xenografts (PDXs). RNA sequencing of EM-treated GBM demonstrated robust modulation of over 1800 genes related to GBM-associated electrical network signaling and GBM-neuronal activity drivers, including the TM-promoting protein GAP-43, NLGN3, excitatory glutamatergic neurotransmission receptors AMPA and NMDA, and a multitude of ion channel signaling and gap junction network communication proteins. This work seeks to build upon those findings to further characterize how modulation of the electrical tumor network impacts GBM pathogenesis and therapeutic sensitivity. It is contemplated that EM applied to GBM-bearing subjects will modulate GBM network-associated signaling proteins, disconnect pathologic GBM intercellular electrical networks, and render GBM exquisitely vulnerable to chemotherapy, immunotherapy, and ionizing radiotherapy.

In one aspect the disclosed systems and methods may use EM to upregulate, downregulate, or otherwise modulate GBM network-associated signaling proteins. For instance, critical network signaling proteins such as GAP-43, NLGN3, AMPA and NMDA receptors, and phosphorylated mTOR (p-mTOR) may be upregulated, downregulated, or otherwise modulated following EM depending on the number of EM sessions delivered and/or the time post-EM.

In another aspect, electrically modulated GBM cells may be functionally disconnected from tumor and neuronal networks. For instance, EM-treated tumors may demonstrate significantly less intratumoral network connections and pathologic integration with neuronal networks.

In another aspect, EM may increase sensitivity to radiation therapy, immunotherapy, and/or chemotherapy. For instance, tumors treated with EM followed by temozolomide (TMZ) chemotherapy, immunotherapy, and/or radiotherapy (RT) may demonstrate more DNA double stranded breaks and decreased clonogenic survival relative to sham-treated tumors, which may translate into prolonged overall survival in subjects.

Disrupting the interactions between neurons and GBM is robustly therapeutic in preclinical models. This work utilizes a novel, translatable electrical mechanism to study these preclinical insights and sensitize GBM to standard-of-care therapy. Traditionally, aggressive central nervous system (CNS) tumors are treated with tri-modality therapy: surgical resection followed by radiotherapy with concurrent and adjuvant chemotherapy. Given recent understanding of the role played by the nervous system in cancer development, progression, and therapeutic resistance, the disclosed systems and methods make therapeutic use of the electrical language spoken by the CNS with a fourth modality: electrical modulation.

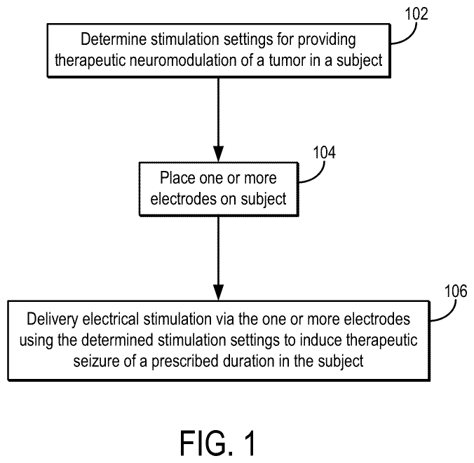

Referring now to FIG. 1, a flowchart is illustrated as setting forth the steps of an example method for treating brain tumor using electrical stimulation or other suitable electrical modulation of the brain tumor to induce a therapeutic seizure in the subject. Although the method is described with respect to providing a therapeutic effect in a glioblastoma, the methods can also be used to provide a therapeutic effect in other brain tumors (e.g., astrocytoma, etc.) or other tumor types.

The method includes determining one or more stimulation settings for providing therapeutic electrical modulation to the subject, as indicated at step 102. The stimulation settings can be predetermined settings that are received from a memory or other data storage device or medium. Additionally or alternatively, the stimulation settings can be generated using a computer system, or selected by a user from a list of preselecting stimulation setting values. In some implementations, determining the stimulation settings may include updating, adjusting, or otherwise revising the stimulation settings for a subject based on feedback data. The feedback data may include user preference data, or may be based on EEG signal data received from the subject before, during, or after delivery of electrical modulation to the subject.

The stimulation settings can include a voltage setting, a current level setting, a frequency setting, a pulse width setting, and a duration setting. As an example, the current level setting can be selected from a range of 20-100 mA. As another example, the frequency setting can be selected from a range of 10-100 Hz. As yet another example, the pulse width can be selected from a range of 0.5-2.0 ms. At still another example, the duration can be selected from a range of 0.5-2.0 s. The stimulation settings are selected to provide a therapeutic effect in the subject by modulating one or more genes associated with a GBM signaling pathway in the subject. In some instances, the stimulation settings are selected to induce a therapeutic seizure in the subject for a prescribed duration of time, thereby providing the therapeutic effect. As a non-limiting example, the stimulation settings can be selected such that the therapeutic seizure in the subject will have a duration of between 10 and 120 seconds. More generally, the therapeutic effect can include modulating (e.g., downregulating, upregulating, both) one or more genes in the subject. The one or more genes may be associated with one or more GBM signaling pathways, such as one or more GBM-associated electrical network signaling pathways, one or more GBM neuronal activity drivers, one or more GBM network-associated signaling proteins, or combinations thereof.

One or more electrodes are placed on the subject, as indicated at step 104. For instance, the electrode(s) may be placed on the surface of the subject's skin near the location to be treated. This may include placing the one or more electrodes on the subject's scalp, or other locations on or near the subject's head, such that electrical modulation can be delivered to one or more target locations in the brain. In some embodiments, the one or more electrodes may include one or more implantable electrodes. For instance, an implantable electrode used for deep brain stimulation, or the like, may be implanted and used to deliver neuromodulation to the subject.

Electrical modulation is then delivered to the subject via the one or more electrodes by controlling a neuromodulation device using the determined stimulation settings to provide a therapeutic effect in the subject, as indicated at step 106. For instance, the controller of the neuromodulation device can receive the stimulation settings (e.g., from a memory of the controller). The controller processes the stimulation settings to control the delivery of electrical modulation via the one or more electrodes. As one example, the provided therapeutic effect can include modulating one or more genes to act on brain tumor networks to slow tumor growth and improve survival in the upfront, adjuvant, and recurrent treatment setting. For instance, the electrical modulation may downregulate, upregulate, or otherwise modulate GBM network-associated signaling proteins to slow tumor growth. As a non-limiting example, network signaling proteins such as GAP43, NLGN3, AMPA receptors, NMDA receptors, and phosphorylated mTOR (p-mTOR) may be downregulated, upregulated, or otherwise modulated by electrical stimulation delivered to the subject according to the stimulation settings.

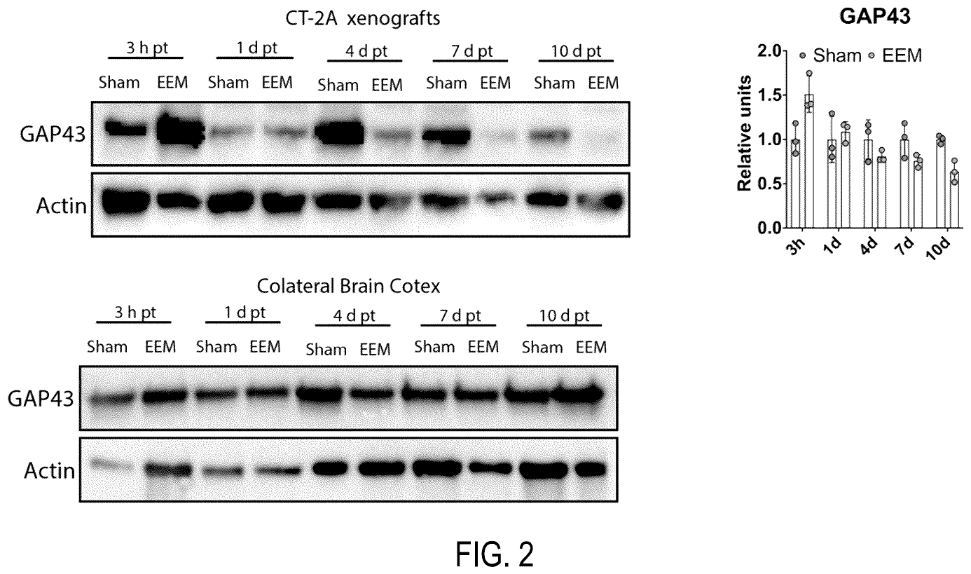

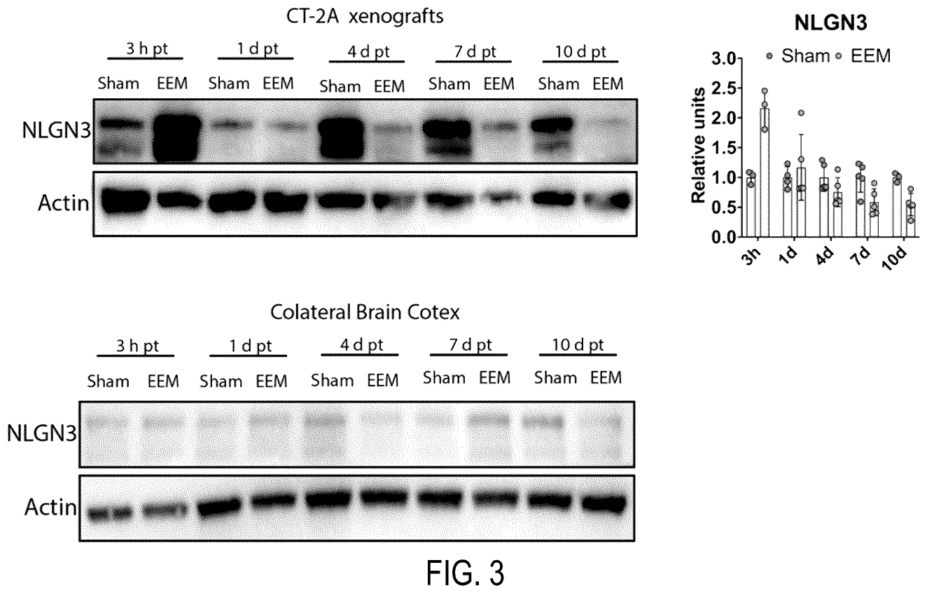

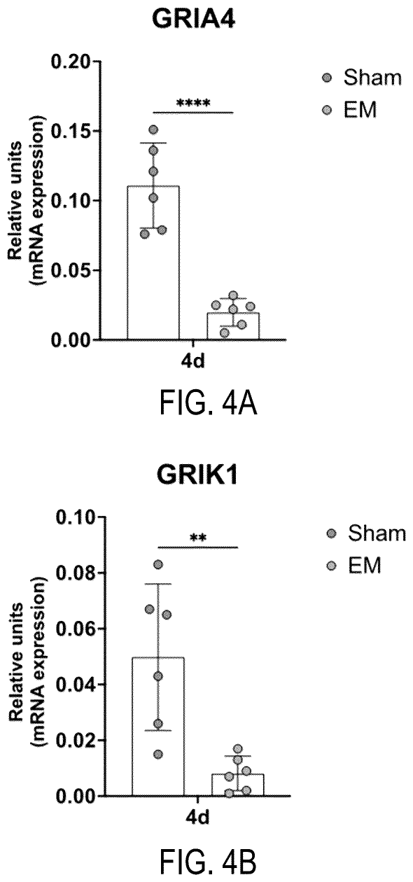

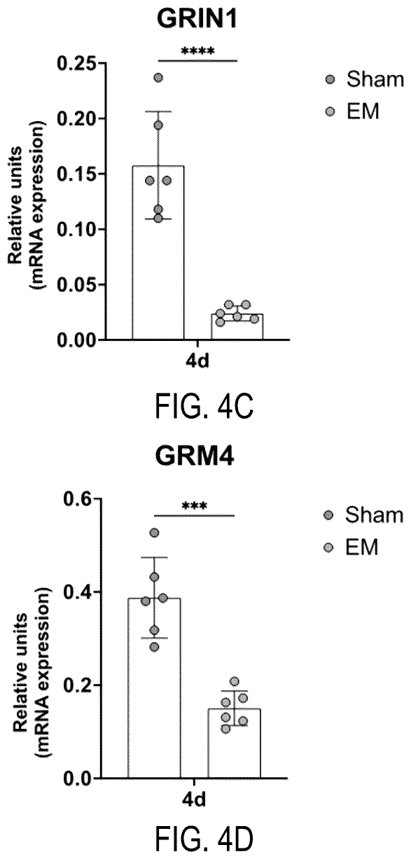

As illustrated in FIG. 2, electrical modulation delivered using the determined stimulation settings can provide a therapeutic effect by downregulating GBM tumor network forming protein neuronal growth associated protein 43 (GAP43). As another example, as illustrated in FIG. 3, electrical modulation delivered using the determined stimulation settings can provide a therapeutic effect by downregulating GBM tumor network forming protein neuroligin 3 (NLGN3). In still other examples, as illustrated in FIGS. 4A-4D, electrical modulation delivered using the determined stimulation settings can provide a therapeutic effect by downregulating glutamate receptors GRIA4 (FIG. 4A), GRIK1 (FIG. 4B), GRIN1 (FIG. 4C), and/or GRM4 (FIG. 4D). The results shown in FIG. 4A-4D were obtained via reverse transcription-quantitative polymerase chain reaction (RT-qPCR).

In some implementations, one or more additional therapies can be administered to the subject before, during, and/or after the electrical modulation is delivered to the subject. For example, the subject may additionally receive radiation therapy, immunotherapy, and/or chemotherapy to complement the neuromodulation provided by the electrical stimulation. The delivered electrical modulation may transiently permit passage of therapeutics across the blood-brain barrier. As one example, chemotherapeutics, immunotherapies, and/or targeted therapies with traditionally poor central nervous system penetrance may be administered to the subject before, during, or after delivery of the electrical modulation. Then, by modulating the blood-brain barrier using the electrical stimulation, the administered therapeutic(s) can cross the blood-brain barrier to achieve an improved therapeutic effect in the subject. Additionally or alternatively, the delivered electrical modulation may sensitize brain tumors to radiation therapy, immunotherapy, and/or chemotherapy. In these instances, radiation therapy, immunotherapy, and/or chemotherapy may be delivered or otherwise administered to the subject before, during, or after delivery of the electrical modulation to provide synergy with and/or enhancing of these current standard-of-care therapeutic options.

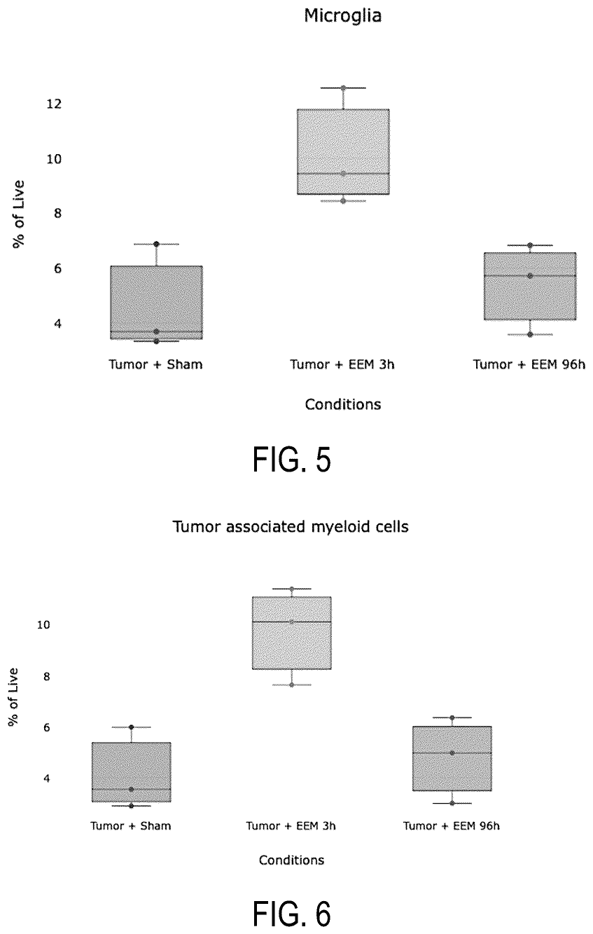

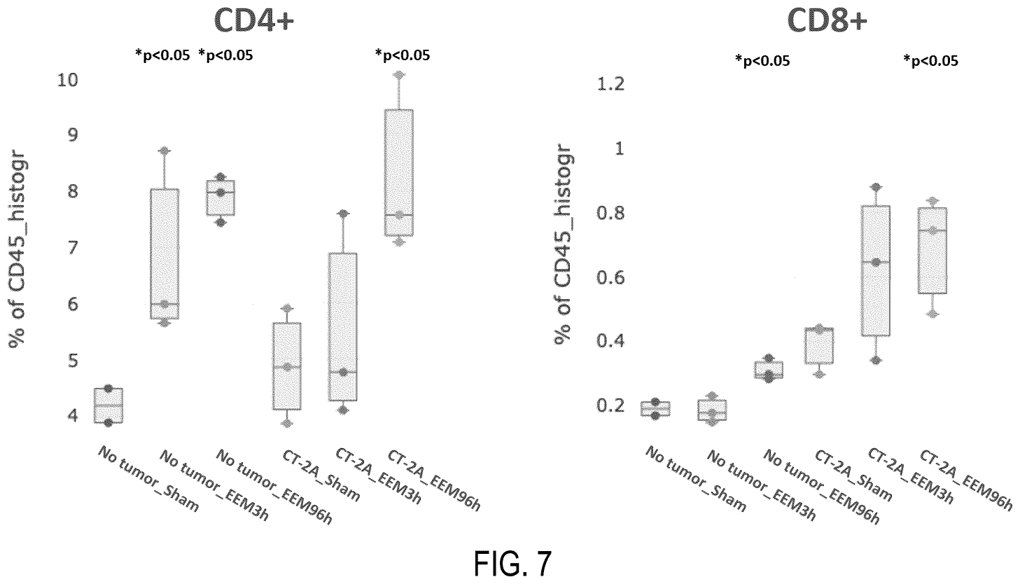

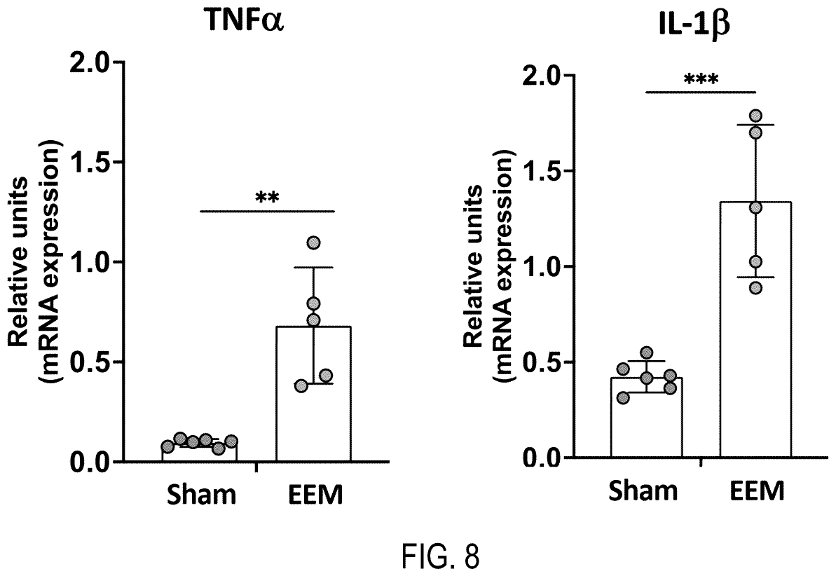

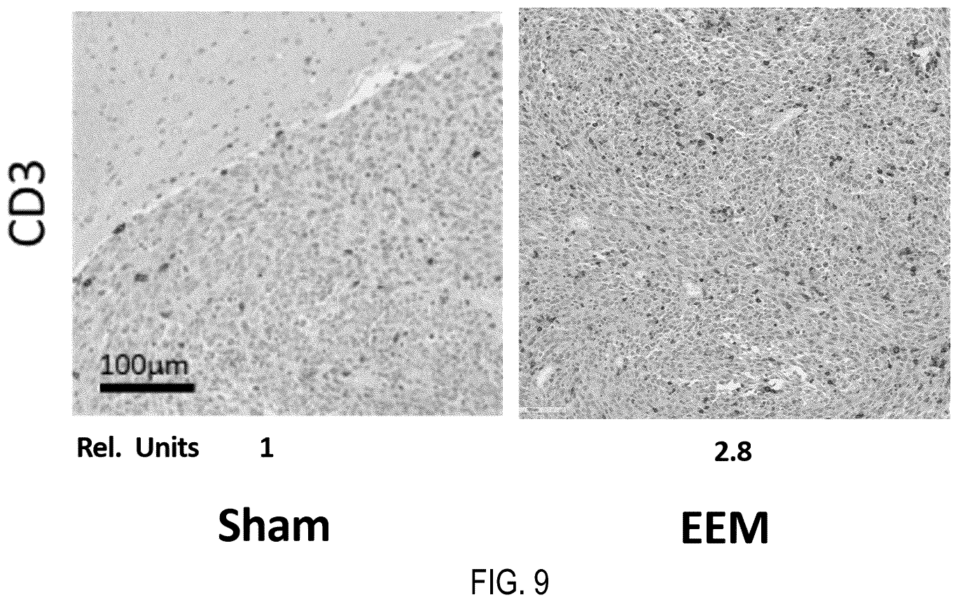

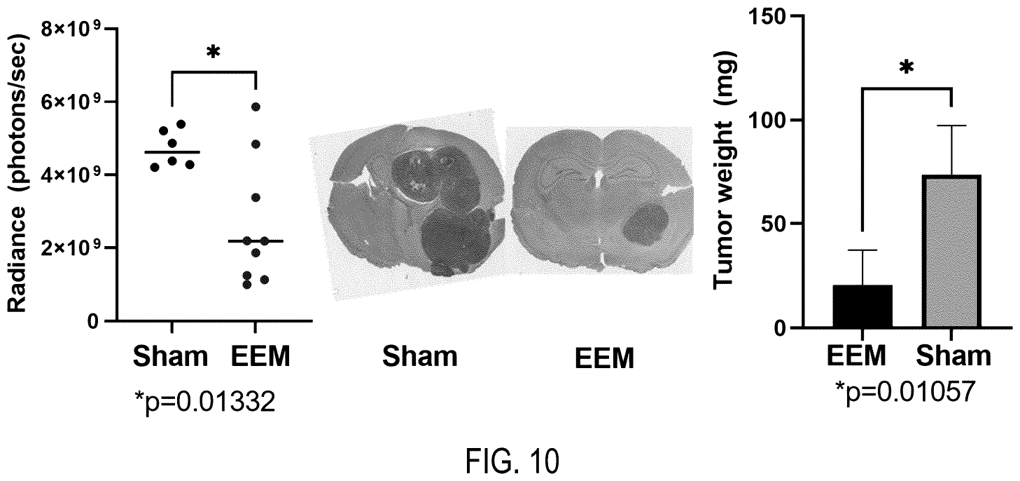

In some example studies, following delivery of electrical modulation using the stimulation settings determined to provide a therapeutic effect, increased intratumor microglia (FIG. 5) and increased intratumoral tumor-associated myeloid cells (FIG. 6) were observed. Additionally, results from example studies indicate that delivery of electrical modulation using the stimulation settings determined to provide a therapeutic effect resulted in increased intratumoral CD4+ and CD8+ T-cells (FIG. 7), increased intratumoral innate inflammatory markers within 24 hours following electrical modulation (FIG. 8), and/or increased intratumoral CD3+ T-cells (FIG. 9). FIG. 10 shows an example of tumor bioluminescence, H&E stained images, and tumor weights after taken before and after delivery of electrical modulation using the stimulation settings determined to provide a therapeutic effect.

In an example study, EM with electrical stimulus sufficient to produce a therapeutic seizure was explored. Prior to evaluating biological effects of EM, a study was conducted to verify that seizures could be reliably produced in mice bearing glioma-xenografts.

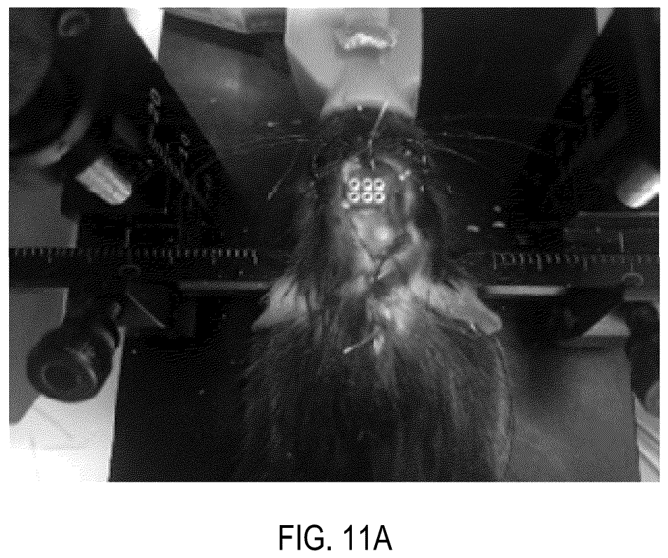

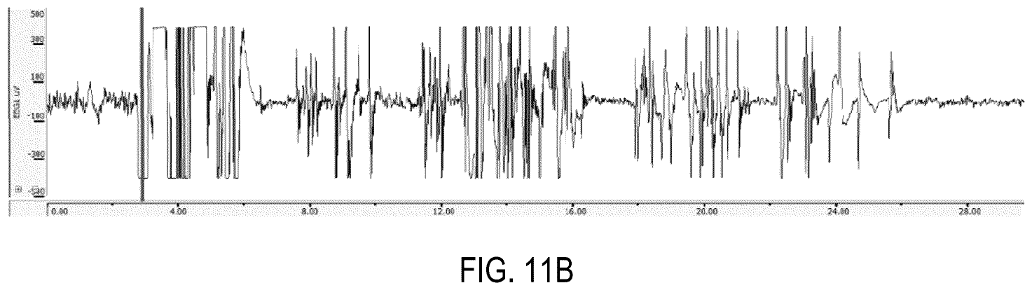

Following orthotopic injection of CT-2A glioma cells and EEG implantation (FIG. 11A), C57BL6 mice underwent EM via ear-clip electrodes delivered with the Ugo Basile model 57800 machine (Stoelting Co.) at a current level of 80 mA, frequency of 50 Hz, pulse width of 0.5 ms, and shock duration of 1.0 s. Seizure activity was reproducibly characterized by a sequence of events starting with whole body arching followed by tonic hindlimb extension and then facial, forelimb, and hindlimb clonus. Seizures clinically lasted an average of 28 seconds (range: 20 to 44 seconds), which was confirmed on EEG readings (FIG. 11B).

After confirmation of reliable seizure induction, the effects of EM on intracranial tumor progression and overall survival in mice bearing glioma-xenografts were determined.

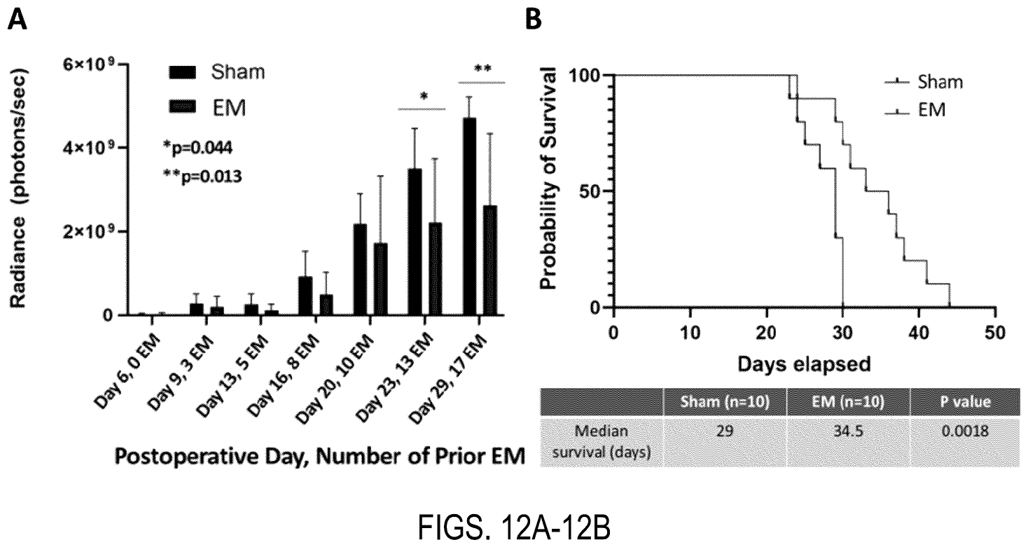

Following orthotopic injection of CT-2A-Luc glioma cells, C57BL6 mice were randomized to receive EM via ear-clip electrodes delivered with the Ugo Basile model 57800 machine (Stoelting Co.) at a current level of 80 mA, frequency of 50 Hz, pulse width of 0.5 ms, and shock duration of 1.0 s or sham treatment daily up to five times per week. Intracranial progression was monitored via bioluminescent signal from CT-2A-Luc xenografts and was maximally reduced in EM-treated mice relative to sham-treated mice after 17 EM treatments (FIG. 12A, EM radiance 2.6×10⁹ photons/second versus sham 4.7×10⁹ photons/second, p=0.013). This translated into an improvement in overall survival from median 29 days in sham-treated mice to 34.5 days in EM-treated mice (FIG. 12B, p=0.0018).

After determining that EM reduces intracranial tumor progression and prolongs overall survival in xenograft-bearing mice, it was determined whether clinical seizures in which mice exhibited bilateral hind limb extension (LE) was associated with overall survival in mice. Seizures with LE were clinically longer in duration and hypothesized to be more effective therapeutically.

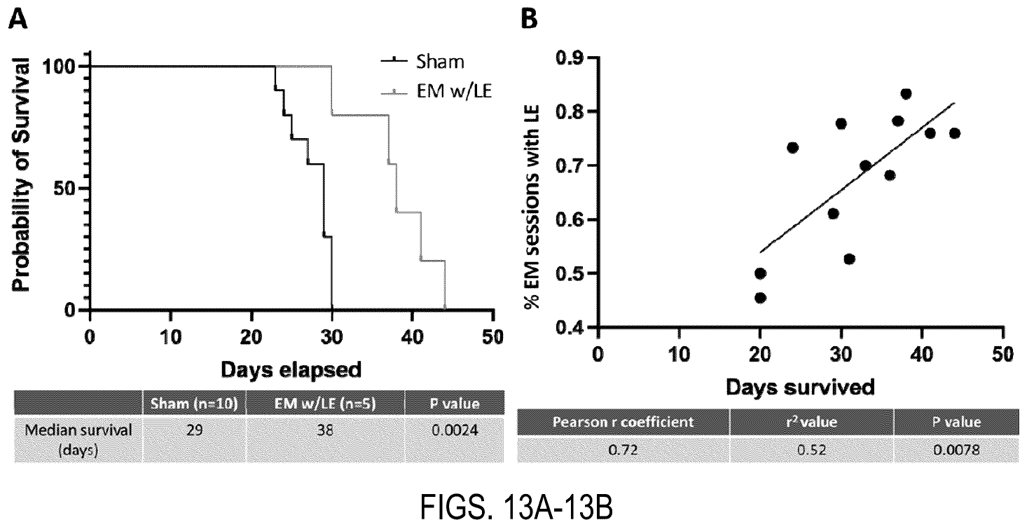

Following orthotopic injection of CT-2A-Luc glioma cells, C57BL6 mice were randomized to receive EM via ear-clip electrodes delivered with the Ugo Basile model 57800 machine (Stoelting Co.) at a current level of 80 mA, frequency of 50 Hz, pulse width of 0.5 ms, and shock duration of 1.0 s or sham treatment daily up to five times per week. Only mice with over 75% percent of clinical seizures including bilateral hind limb extension were included for analysis and experienced improvement in overall survival from median 29 days in sham-treated mice to 38 days in EM w/LE-treated mice (FIG. 13A, p=0.0024). The percent of EM sessions with bilateral hind limb extension was significantly correlated with survival (FIG. 13B, Pearson r coefficient=0.72, r² value=0.52, p=0.0078).

As relevant for therapeutic synergy with chemotherapy, it was also evaluated whether EM produces increased permeability of the BBB in C57BL6 mice without glioma xenografts.

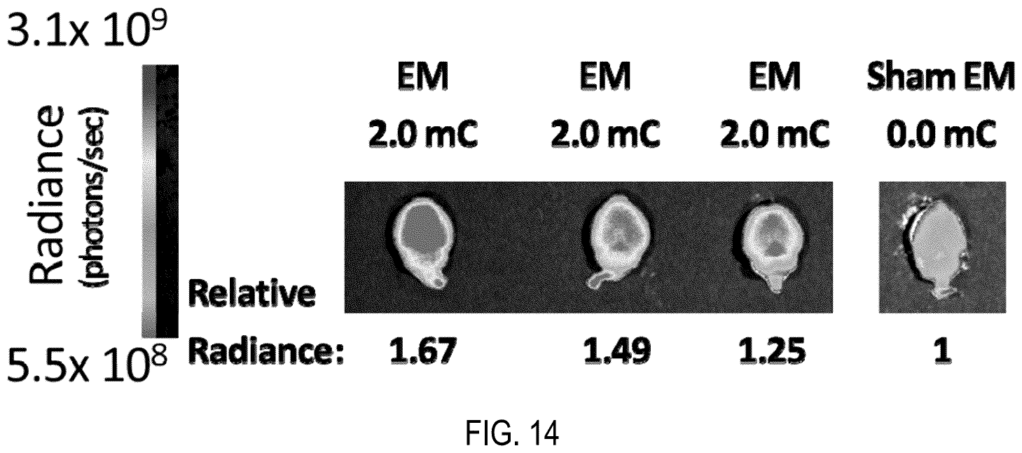

Two groups of healthy C57BL6 mice were tail vein injected with sodium fluorescein (40 mg/kg mouse). Approximately five minutes following injection, mice were randomized to either a single EM session delivered via ear-clip electrodes with the Ugo Basile model 57800 machine (Stoelting Co.) at a current level of 80 mA, frequency of 50 Hz, pulse width of 0.5 ms, and shock duration of 1.0 s or sham treatment. Approximately 30 minutes after tail vein injection, all mice were euthanized and perfused with phosphate buffered saline (PBS) solution. Brains were harvested and evaluated for fluorescence on the IVIS machine at excitation wavelength of 465 nm and emission wavelength of 530 nm. Relative radiance was increased across all EM-treated mice by up to 1.67 relative to sham EM-treated mice (FIG. 14).

To begin exploring the mechanism of prolonged survival in mice treated with EM, RNA sequencing of xenografted CT-2A tumors from C57BL6 mice treated with 13 sessions of EM or sham (n=3 mice per group) was also performed.

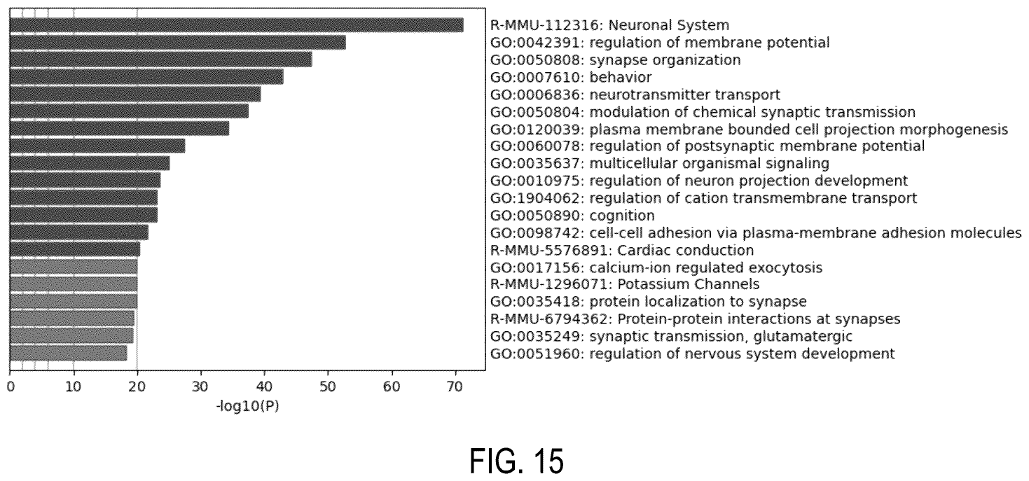

In all, 1960 genes were statistically different between groups, with 1849 genes downregulated and 111 genes upregulated. Gene ontological analysis of statistically downregulated genes demonstrated highly significant and clustered downregulation of tumor-related neuronal activity, synaptic organization, neurotransmission including glutamatergic signaling, potassium channel signaling, and gap junction signaling (FIG. 15).

As relevant to the disclosed systems and methods, the notable downregulated genes and their role in GBM pathogenesis are highlighted in Table 1.

TABLE 1
Notable Downregulated Tumor Genes by RNA Sequencing

| Gene | | | Log2-Fold | |
|---|---|---|---|---|
| Symbol | Gene | Role in GBM Network | Change | P-value |
| GAP43 | Neuronal growth | Gap junction protein; TM- | −2.7 | 0.0055 |
| | Associated protein 43 | dependent invasion, | | |
| | | proliferation, and | | |
| | | therapeutic resistance | | |
| NLGN3 | Neuroligin 3 | Synaptic protein required | −4.4 | 0.000036 |
| | | for GBM progression | | |
| GRIA4 | Glutamate Receptor, | Glutamatergic receptor | −6.6 | 8.4 × 10⁻⁹ |
| | Ionotropic, AMPA4 | that fuels GBM | | |
| | | progression | | |
| GRIN2C | Glutamate Receptor, | Glutamatergic receptor | −6.0 | 5.6 × 10⁻⁷ |
| | Ionotropic, | that fuels malignant brain | | |
| | NMDA2C | tumor progression and | | |
| | | radioresistance | | |
| PREX2 | Phosphatidylinositol- | Activates RAC (cancer | −2.6 | 0.00068 |
| | 3,4,5- | cell invasion) downstream | | |
| | trisphosphatedependent | of PI3K-mTOR pathway | | |
| | Rac exchange factor 2 | | | |
| KCNJ3 | Potassium inwardly- | Inward Potassium Current | −7.3 | 2.0 × 10⁻⁹ |
| | rectifying | Utilized by GBM | | |
| | channel | | | |
| NRXI | Neurexin I | Synaptic cleft protein that | −6.4 | 1.1 × 10⁻⁸ |
| | | binds neuroligin | | |
| SLC1A2 | Solute carrier family | Glutamate transporter that | −5.9 | 1.8 × 10⁻⁷ |
| | 1 (glutamate | is upregulated by | | |
| | transporter) | neuronally-stimulated | | |
| | | GBM cells | | |
| GJC3 | Gap junction protein, | Gap junction protein that | −5.3 | 1.7 × 10⁻⁷ |
| | gamma 3 | facilitates intercellular | | |
| | | communication | | |

The 111 statistically upregulated genes did not cluster as closely as the downregulated genes, but of note, three of the top five most significant genes were granzyme D, F, and G, which could indicate an increase in immune-related cytolysis.

In summary, EM slows intracranial progression and prolongs overall survival in glioma xenograft-bearing mice while also increasing BBB permeability. RNA sequencing of EM-treated tumors demonstrates significant downregulation of over 1800 genes related to GBM-associated neuronal activity and electrical network signaling, including the TM-promoting protein GAP-43, NLGN3, excitatory glutamatergic neurotransmission receptors AMPA and NMDA, inward potassium current channels, and gap junction network proteins.

Growing evidence indicates that the neurotransmitters dysregulated in psychiatric disorders are similarly dysregulated in glioblastoma (GBM) biology. Patients with psychiatric disorders are classically thought of having excessive glutamate (major depression, bipolar depression), excessive dopamine (mania, psychosis), and/or insufficient serotonin (major depression, anxiety). Analogously, GBM cells are dependent on bountiful neuronal glutamate, utilize elevated dopamine receptor expression to activate the hypoxic response and augment progression, and catabolize serotonin to drive proliferation. The clinical induction of seizure, known as electroconvulsive therapy (ECT), has been used by psychiatrists since the 1930s to treat severe cases of depression, mania, psychosis, and catatonia. ECT has been shown to decrease extracellular glutamate and glutamate receptor expression in vivo and increase blood-brain barrier (BBB) permeability. ECT, then, may have previously unexplored oncologic value.

It was hypothesized that seizure-induced changes in the glioma microenvironment occur with ECT, increasing permeability of the BBB, slowing tumor progression, and prolonging overall survival in glioma-bearing mice.

C57BL6 mice were orthotopically injected with CT-2A-Luc mouse glioma cells. Mice were randomized to receive ECT via ear-clip electrodes or sham treatment daily up to five times per week until survival endpoints were reached. Seizure duration and rates of tonic hindlimb extension were recorded. Intracranial progression was monitored via bioluminescent signal from CT-2A-Luc xenografts. BBB permeability was assessed by subjecting mice to ECT or sham treatment immediately following tail vein injection of sodium fluorescein. Mice were saline perfused and brains were harvested for fluorescent spectroscopy.

Intracranial progression was maximally reduced in ECT-treated mice relative to sham-treated mice after 17 treatments (mean sham radiance 4.7×10⁹ photons/s versus mean ECT radiance 2.6×10⁹ photon/s, P=0.013). This translated into an improvement in overall survival from median 29 days in sham-treated mice to 34.5 days in ECT-treated mice (P=0.0018). Mean seizure duration was 41.8 seconds and positively correlated with overall survival (Pearson coefficient r=0.63, P=0.028). Tonic hindlimb extension occurred in 68% of seizures and positively correlated with overall survival (Pearson coefficient r=0.72, P=0.0078). Brain parenchymal uptake of sodium fluorescein was significantly higher in ECT-treated mice, with a mean relative increase in ECS to sham radiance of 1.47 (P<0.05).

Repeated ECT slows tumor progression and prolongs overall survival in C57BL6 mice bearing CT-2A-Luc xenografts. The BBB is compromised immediately following ECT. ECT merits further oncologic investigation as a potential therapeutic in GBM.

In an example study, mice were orthotopically injected with 1×10⁵ tumor cells on day 0. This was followed by EM or sham procedure on days 7, 9, and 11. Radiation therapy (or sham radiation therapy) and chemotherapy (e.g., using temozolomide (TMZ) or vector OraPlus) began concurrently on day 8 and continued daily through day 12. TMZ was be delivered by oral gavage.

On day 13 (24 hours post-last TMZ and radiation therapy), a cohort of mice was euthanized and tumors were harvested for gamma H2AX staining and comet assay analysis for DNA double stranded breaks. A clonogenic survival analysis was performed on dissociated xenografts.

It was demonstrated that tumors treated with EM followed by TMZ chemotherapy and/or radiation therapy demonstrated more DNA double stranded breaks and decreased clonogenic survival relative to sham-treated tumors, which will translate into prolonged overall survival.

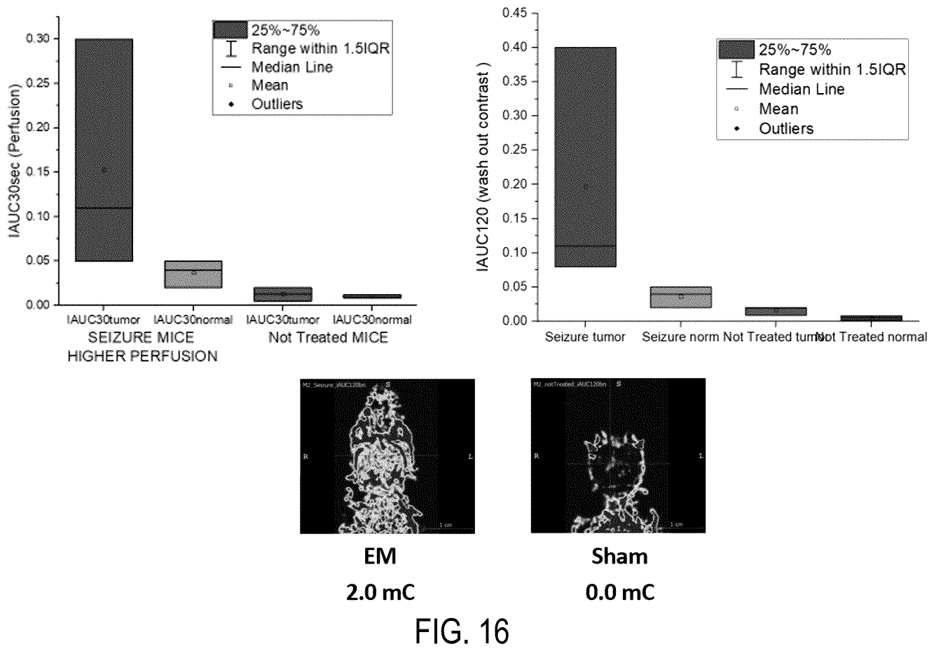

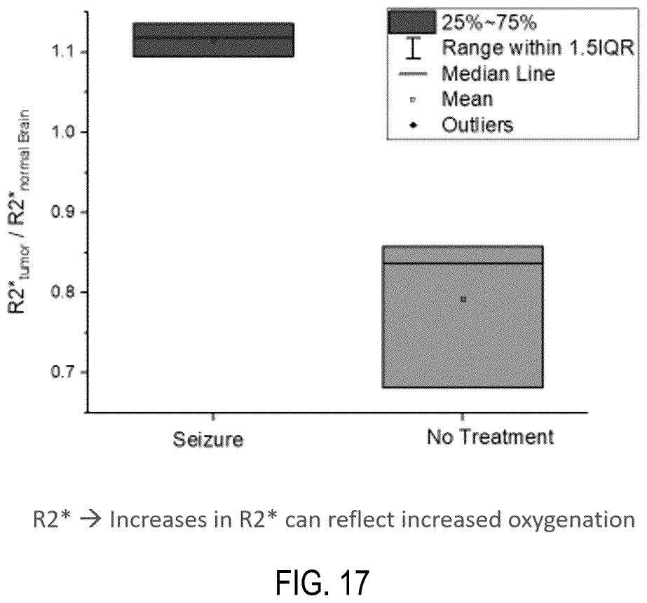

It was also observed, as shown in FIG. 16, that increased perfusion after EM was measurable using multiparametric magnetic resonance imaging (MRI). This increased perfusion is another indication of increased sensitivity to radiation. Additionally, increased oxygenation after EM was also observed via multiparametric MRI, as illustrated in FIG. 17. For instance, the increased measurements of R2* indicate an increase in oxygenation following EM. This increased oxygenation is yet another indication of increased sensitivity to radiation.

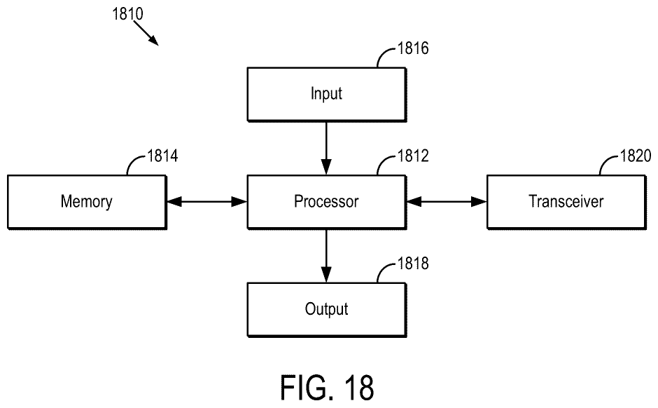

Referring now to FIG. 18, an example of a controller 1810 that can implement the methods described in the present disclosure to control a neuromodulation device, such as a brain stimulation device, is illustrated. In general, the controller 1810 includes a processor 1812, a memory 1814, and input 1816, and an output 1818. The controller 1810 can be implemented as part of a neuromodulation device, or as a separate controller that is in communication with the neuromodulation device via the output 1818. As one example, the controller 1810 can be implemented in a neuromodulation device, such as an implantable medical device (e.g., an implanted nerve stimulation system), a standalone neuromodulation device (e.g., an externally applied electrical and/or magnetic stimulation device), and so on. In other examples, the controller 1810 can be implemented in a remote computer that communicates with the neuromodulation device. In still other examples, the controller 1810 can be implemented in a smartphone that is paired with the neuromodulation device, such as via Bluetooth or another wireless or wired communication.

In some embodiments, the input 1816 is capable of recording neural signal data, or other physiological measurement data, from the user. As one example, the neural signal data can be electrophysiological activity data (e.g., EEG signal data), and the input 1816 can be one or more electrodes. The input 1816 can include a wired or wireless connector for receiving neural signal data. These neural signal data can be transmitted to the controller 1810 via the input 1816.

The processor 1812 includes at least one hardware processor to execute instructions embedded in or otherwise stored on the memory 1814 to implement the methods described in the present disclosure. The memory can also store baseline signal data, measured neural signal data, one or more models, and model parameters, as well as settings to be provided to the processor 1812 for generating control signals to be provided to a neuromodulation device via the output 1818.

The output 1818 communicates control signals to a neuromodulation device, which may be an electrical modulation device. As one example, where the neuromodulation device is an electrical modulation device including one or more electrodes, the control signals provided to the output 1818 can control one or more electrodes to operate under control of the controller 1810 to deliver electrical stimulations, or other electrical modulation, to the subject. The control signals may include electrical stimulation parameters such as voltage, current level, frequency, pulse width, and shock duration.

The controller 1810 can also include a transceiver 1820 and associated circuitry for communicating with a programmer or other external or internal device. As one example, the transceiver 1820 can include a telemetry coil. In some embodiments, the transceiver 1820 can be a part of the input 1816.

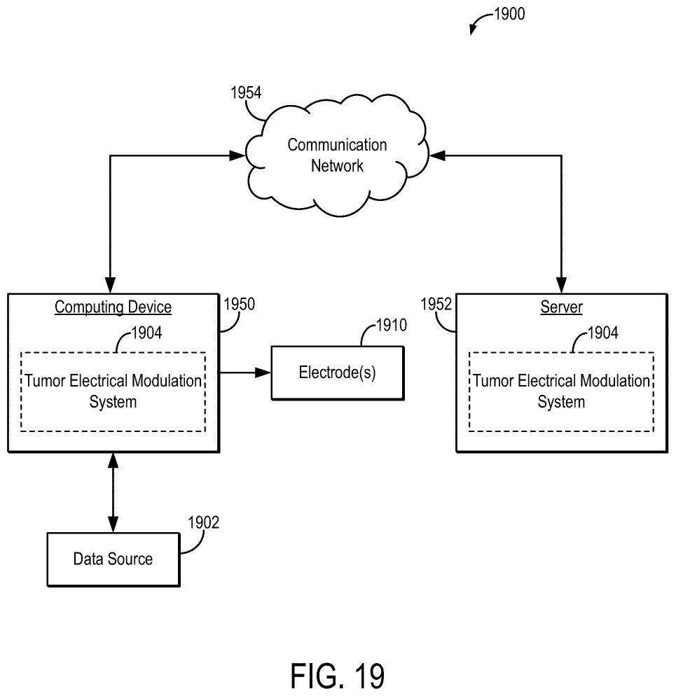

FIG. 19 shows an example of a system 1900 for providing a therapeutic effect in tumors using electrical modulation in accordance with some embodiments described in the present disclosure. As shown in FIG. 19, a computing device 1950 can receive one or more types of data (e.g., stimulation settings, EEG signal data, etc.) from data source 1902. In some embodiments, computing device 1950 can execute at least a portion of a tumor electrical modulation system 1904 to deliver electrical modulation to a subject based on stimulation settings received from the data source 1902.

Additionally or alternatively, in some embodiments, the computing device 1950 can communicate information about data received from the data source 1902 to a server 1952 over a communication network 1954, which can execute at least a portion of the tumor electrical modulation system 1904. In such embodiments, the server 1952 can return information to the computing device 1950 (and/or any other suitable computing device) indicative of an output of the tumor electrical modulation system 1904.

In some embodiments, computing device 1950 and/or server 1952 can be any suitable computing device or combination of devices, such as a desktop computer, a laptop computer, a smartphone, a tablet computer, a wearable computer, a server computer, a virtual machine being executed by a physical computing device, and so on. In some embodiments, the computing device 1950 may be the processor 1812 of the neurostimulation device controller 1810. The computing device 1950 processes the stimulation settings received from the data source 1902 and/or determined (e.g., generated, selected, adjusted, updated) by the computing device 1950 to generate control signals for operating one or more electrodes 1910 to deliver electrical stimulation to the subject. For instance, the control signals can control the electrical stimulation generated by the electrode(s) 1910 such that electrical modulation of a targeted tumor is provided.

In some embodiments, data source 1902 can be any suitable source of data (e.g., measurement data, stimulation settings), another computing device (e.g., a server storing measurement data, stimulation settings), and so on. In some embodiments, data source 1902 can be local to computing device 1950. For example, data source 1902 can be incorporated with computing device 1950 (e.g., computing device 1950 can be configured as part of a device for measuring, recording, estimating, acquiring, or otherwise collecting or storing data). As another example, data source 1902 can be connected to computing device 1950 by a cable, a direct wireless link, and so on. Additionally or alternatively, in some embodiments, data source 1902 can be located locally and/or remotely from computing device 1950, and can communicate data to computing device 1950 (and/or server 1952) via a communication network (e.g., communication network 1954).

In some embodiments, communication network 1954 can be any suitable communication network or combination of communication networks. For example, communication network 1954 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 1954 can be a local area network, a wide area network, a public network (e.g., the Internet), a private or semi-private network (e.g., a corporate or university intranet), any other suitable type of network, or any suitable combination of networks. Communications links shown in FIG. 19 can each be any suitable communications link or combination of communications links, such as wired links, fiber optic links, Wi-Fi links, Bluetooth links, cellular links, and so on.

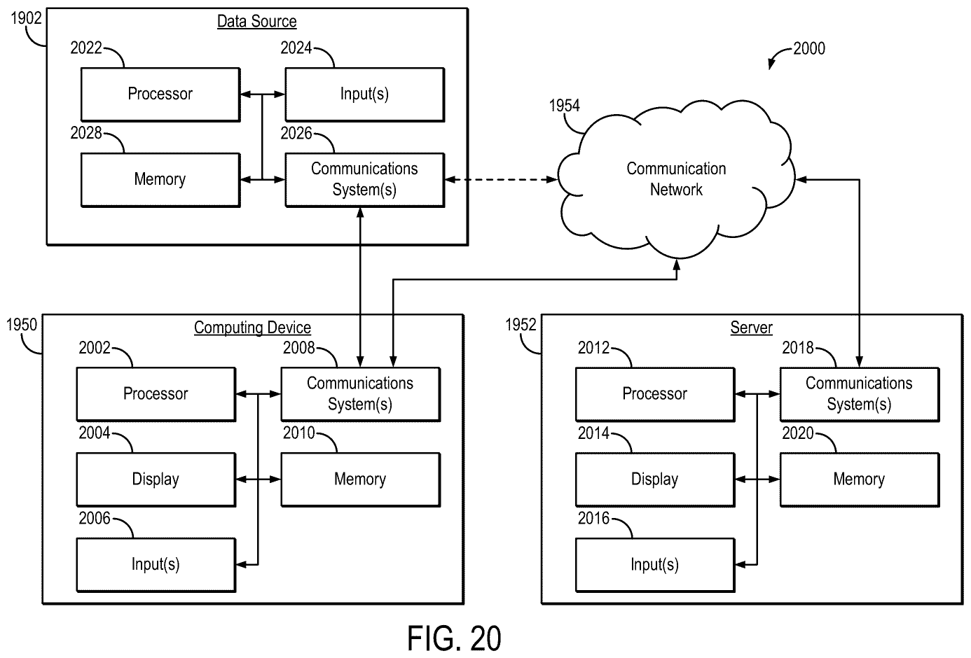

Referring now to FIG. 20, an example of hardware 2000 that can be used to implement data source 1902, computing device 1950, and server 1952 in accordance with some embodiments of the systems and methods described in the present disclosure is shown.

As shown in FIG. 20, in some embodiments, computing device 1950 can include a processor 2002, a display 2004, one or more inputs 2006, one or more communication systems 2008, and/or memory 2010. In some embodiments, processor 2002 can be any suitable hardware processor or combination of processors, such as a central processing unit (CPU), a graphics processing unit (GPU), and so on. In some embodiments, display 2004 can include any suitable display devices, such as a liquid crystal display (LCD) screen, a light-emitting diode (LED) display, an organic LED (OLED) display, an electrophoretic di splay (e.g., an “e-ink” display), a computer monitor, a touchscreen, a television, and so on. In some embodiments, inputs 2006 can include any suitable input devices and/or sensors that can be used to receive user input, such as a keyboard, a mouse, a touchscreen, a microphone, and so on.

In some embodiments, communications systems 2008 can include any suitable hardware, firmware, and/or software for communicating information over communication network 1954 and/or any other suitable communication networks. For example, communications systems 2008 can include one or more transceivers, one or more communication chips and/or chip sets, and so on. In a more particular example, communications systems 2008 can include hardware, firmware, and/or software that can be used to establish a Wi-Fi connection, a Bluetooth connection, a cellular connection, an Ethernet connection, and so on.

In some embodiments, memory 2010 can include any suitable storage device or devices that can be used to store instructions, values, data, or the like, that can be used, for example, by processor 2002 to present content using display 2004, to communicate with server 1952 via communications system(s) 2008, and so on. Memory 2010 can include any suitable volatile memory, non-volatile memory, storage, or any suitable combination thereof. For example, memory 2010 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 2010 can have encoded thereon, or otherwise stored therein, a computer program for controlling operation of computing device 1950. In such embodiments, processor 2002 can execute at least a portion of the computer program to present content (e.g., images, user interfaces, graphics, tables), receive content from server 1952, transmit information to server 1952, and so on. For example, the processor 2002 and the memory 2010 can be configured to perform the methods described herein (e.g., the method of FIG. 1).

In some embodiments, server 1952 can include a processor 2012, a display 2014, one or more inputs 2016, one or more communications systems 2018, and/or memory 2020. In some embodiments, processor 2012 can be any suitable hardware processor or combination of processors, such as a CPU, a GPU, and so on. In some embodiments, display 2014 can include any suitable display devices, such as an LCD screen, LED display, OLED display, electrophoretic display, a computer monitor, a touchscreen, a television, and so on. In some embodiments, inputs 2016 can include any suitable input devices and/or sensors that can be used to receive user input, such as a keyboard, a mouse, a touchscreen, a microphone, and so on.

In some embodiments, communications systems 2018 can include any suitable hardware, firmware, and/or software for communicating information over communication network 1954 and/or any other suitable communication networks. For example, communications systems 2018 can include one or more transceivers, one or more communication chips and/or chip sets, and so on. In a more particular example, communications systems 2018 can include hardware, firmware, and/or software that can be used to establish a Wi-Fi connection, a Bluetooth connection, a cellular connection, an Ethernet connection, and so on.

In some embodiments, memory 2020 can include any suitable storage device or devices that can be used to store instructions, values, data, or the like, that can be used, for example, by processor 2012 to present content using display 2014, to communicate with one or more computing devices 1950, and so on. Memory 2020 can include any suitable volatile memory, non-volatile memory, storage, or any suitable combination thereof. For example, memory 2020 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 2020 can have encoded thereon a server program for controlling operation of server 1952. In such embodiments, processor 2012 can execute at least a portion of the server program to transmit information and/or content (e.g., data, images, a user interface) to one or more computing devices 1950, receive information and/or content from one or more computing devices 1950, receive instructions from one or more devices (e.g., a personal computer, a laptop computer, a tablet computer, a smartphone), and so on.

In some embodiments, the server 1952 is configured to perform the methods described in the present disclosure. For example, the processor 2012 and memory 2020 can be configured to perform the methods described herein (e.g., the method of FIG. 1).

In some embodiments, data source 1902 can include a processor 2022, one or more inputs 2024, one or more communications systems 2026, and/or memory 2028. In some embodiments, processor 2022 can be any suitable hardware processor or combination of processors, such as a CPU, a GPU, and so on. In some embodiments, the one or more inputs 2024 are generally configured to receive data (e.g., user inputs, predetermined stimulation settings, etc.). Additionally or alternatively, in some embodiments, the one or more inputs 2024 can include any suitable hardware, firmware, and/or software for coupling to and/or controlling operations of electrodes, etc. In some embodiments, one or more portions of the input(s) 2024 can be removable and/or replaceable.

Note that, although not shown, data source 1902 can include any suitable inputs and/or outputs. For example, data source 1902 can include input devices and/or sensors that can be used to receive user input, such as a keyboard, a mouse, a touchscreen, a microphone, a trackpad, a trackball, and so on. As another example, data source 1902 can include any suitable display devices, such as an LCD screen, an LED display, an OLED display, an electrophoretic display, a computer monitor, a touchscreen, a television, etc., one or more speakers, and so on.

In some embodiments, communications systems 2026 can include any suitable hardware, firmware, and/or software for communicating information to computing device 1950 (and, in some embodiments, over communication network 1954 and/or any other suitable communication networks). For example, communications systems 2026 can include one or more transceivers, one or more communication chips and/or chip sets, and so on. In a more particular example, communications systems 2026 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 2028 can include any suitable storage device or devices that can be used to store instructions, values, data, or the like, that can be used, for example, by processor 2022 to control the one or more inputs 2024, and/or receive data from the one or more inputs 2024; to generate images from data; present content (e.g., data, images, a user interface) using a display; communicate with one or more computing devices 1950; and so on. Memory 2028 can include any suitable volatile memory, non-volatile memory, storage, or any suitable combination thereof. For example, memory 2028 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 2028 can have encoded thereon, or otherwise stored therein, a program for controlling operation of data source 1902. In such embodiments, processor 2022 can execute at least a portion of the program to generate images, transmit information and/or content (e.g., data, images, a user interface) to one or more computing devices 1950, receive information and/or content from one or more computing devices 1950, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for electrical modulation of a glioblastoma (GBM) in a subject, the method comprising:
receiving, by a neuromodulation device, stimulation settings determined to generate an electrical stimulation that modulates one or more genes associated with a GBM signaling pathway; and
delivering, via one or more electrodes connected to the neuromodulation device, the electrical stimulation to the subject using the stimulation settings, wherein the electrical stimulation provides a therapeutic effect in the subject by modulating the one or more genes associated with the GBM signaling pathway.

2. The method of claim 1, wherein the stimulation settings include at least one of a voltage, a current level, a frequency, a pulse width, or a duration of the electrical stimulation.

3. The method of claim 2, wherein the stimulation settings include the current level and the current level is selected from a range of 20-100 mA.

4. The method of claim 2, wherein the stimulation settings include the frequency and the frequency is selected from a range of 10-100 Hz.

5. The method of claim 2, wherein the stimulation settings include the pulse width and the pulse width is selected from a range of 0.5-2.0 ms.

6. The method of claim 2, wherein the stimulation settings include the duration of the electrical stimulation and the duration of the electrical stimulation is selected from a range of 0.5-2.0 s.

7. The method of claim 1, further comprising providing at least one additional treatment before, during, or after the electrical stimulation.

8. The method of claim 7, wherein the at least one additional treatment is at least one of radiation therapy, immunotherapy, or chemotherapy.

9. The method of claim 1, wherein the stimulation settings are predetermined to induce a therapeutic seizure in the subject having a duration of between 10 and 120 seconds, such that the therapeutic seizure induces modulating the one or more genes associated with the GBM signaling pathway.

10. The method of claim 1, wherein the GBM signaling pathway comprises one or more GBM-associated electrical network signaling pathways.

11. The method of claim 1, wherein the GBM signaling pathway comprises one or more GBM neuronal activity drivers.

12. The method of claim 1, wherein the GBM signal pathway comprises one or more GBM network-associated signaling proteins.

13. The method of claim 12, wherein the one or more genes comprise at least one of neuronal growth associated protein 43 (GAP43) or neuroligin 3 (NLGN3).

14. The method of claim 1, wherein the one or more genes comprise at least one of GRIA4, GRIK1, GRIN1, or GRM4.

15. The method of claim 1, wherein modulating the one or more genes comprises downregulating the one or more genes.

16. A system for electrical modulation of brain tumors, the system comprising:
one or more electrodes to deliver electrical stimulation to a subject;
a memory having stored thereon electrical stimulation parameters predetermined to induce a therapeutic seizure in a subject having a duration between 10 and 120 seconds; and
a processor to drive the one or more electrodes using the electrical stimulation parameters stored on the memory.

17. The system of claim 16, wherein the electrical stimulation parameters include at least one of a voltage, current level, frequency, pulse width, or duration.

18. The system of claim 17, wherein the current level is selected from a range of 20-100 mA.

19. The system of claim 17, wherein the frequency is selected from a range of 10-100 Hz.

20. The system of claim 17, wherein the pulse width is selected from a range of 0.5-2.0 ms.

21. The system of claim 17, wherein the duration is selected from a range of 0.5-2.0 s.

22. The system of claim 17, wherein the electrical stimulation parameters include at least two of a current level selected from a range of 20-100 mA, a frequency selected from a range of 10-100 Hz, a pulse width selected from a range of 0.5-2.0 ms, and a duration selected from a range of 0.5-2.0 s.
